(19) United States
(12) Patent Application Publication
(10) Pub. No.: US 2025/0270245 A1
(43) Pub. Date: Aug. 28, 2025

(54) CHEMICAL METHODS FOR MAKING RHAMNOLIPID HYDROLYSATE

(71) Applicant: The Procter & Gamble Company, Cincinnati, OH (US)

(72) Inventors: John August WOS, Mason, OH (US); Hilda Andamiche NAMANJA-MAGLIANO, Loveland, OH (US); Victor Manuel ARREDONDO, Maderia, OH (US); Dakota James BROCK, West Chester, OH (US); Lijuan LI, Lebanon, OH (US)

(21) Appl. No.: 19/062,635

(22) Filed: Feb. 25, 2025

Related U.S. Application Data
(60) Provisional application No. 63/735,400, filed on Dec. 18, 2024.
(60) Provisional application No. 63/712,718, filed on Oct. 28, 2024.
(60) Provisional application No. 63/558,670, filed on Feb. 28, 2024.
(60) Provisional application No. 63/557,812, filed on Feb. 26, 2024.

(51) Int. Cl. C07H 15/06; A61K 8/60; A61K 47/26; A61Q 19/00; C07H 1/00

(52) U.S. Cl. CPC C07H 15/06; A61K 8/604; A61K 47/26; A61Q 19/00; C07H 1/00; A61K 2800/10

(57) ABSTRACT

A method of making a rhamnomonolipid hydrolysate by the following steps: a. providing rhamnodilipids; b. treating the rhamnodilipids with a composition comprising a divalent base at a temperature sufficient to cause hydrolysis; c. producing a rhamnomonolipid hydrolysate and by-products; and d. optionally separating the rhamnomonolipid hydrolysate from the by-products.

### CROSS REFERENCE TO RELATED APPLICATIONS

This application claims the benefit, under 35 U.S.C. § 119 (e), to U.S. Provisional Application Nos. 63/735,400 filed Dec. 18, 2024, 63/712,718 filed Oct. 28, 2024, 63/558,670 filed Feb. 28, 2024, and 63/557,812, filed Feb. 26, 2024, the entire disclosure of which is fully incorporated by reference herein.

### FIELD OF THE INVENTION

The present disclosure generally relates to a process for making tailored rhamnolipids through base catalyzed hydrolysis.

### BACKGROUND OF THE INVENTION

Consumers are seeking more natural and milder cleansers without performance trade-offs. Rhamnolipids, a subset of glycolipids, are known and can fulfill certain consumer needs by offering natural surfactants while still providing foaming and lathering. However, commercial rhamnolipids tend to be costly, and many are still low performing compared to current materials. Hence, there is a continuing need for tailored rhamnolipid mixtures of congeners that can provide improved efficacy and consumer experience, and chemical processes that can produce these tailored rhamnolipids.

### SUMMARY OF THE INVENTION

A method of making a rhamnomonolipid hydrolysate by the following steps:
a. providing rhamnodilipids;
b. treating the rhamnodilipids with a composition comprising a divalent base at a temperature sufficient to cause hydrolysis;
c. producing a rhamnomonolipid hydrolysate and by-products; and
d. optionally separating the rhamnomonolipid hydrolysate from the by-products.

### BRIEF DESCRIPTION OF THE FIGURES

FIG. 1 shows an exemplary route for synthesizing rhamno-mono-lipids.

FIG. 2 shows exemplary rhamnolipid structures.

FIG. 3 shows two potential chelation modes for divalent cations.

FIG. 4 shows a schematic of Inventive Example 1.

### DETAILED DESCRIPTION OF THE INVENTION

There is interest in non-traditional surfactants, such as glycolipid biosurfactants, as many palm- and petroleum-based surfactants suffer from sustainability, environmental, and socioeconomic challenges. Glycolipids consist of a diverse group of naturally occurring surfactant molecules with a range of structures (made up of a sugar polar group and a lipid group). The two main commercial classes of glycolipids are rhamnolipids (produced via bacteria plus fermentation) and sophorolipids (produced via yeast fermentation of mixed oil and sugar feed). In addition to being seen as environmentally friendly chemicals and enabling green credentialling, these materials have many other potential benefits such as mildness, moisturization, and cleaning effectiveness.

Many current commercial glycolipids have poor performance and high costs compared to current surfactants. Knowing the structures of certain high-performing glycolipids, the present inventors sought to produce optimized glycolipids. The inventors hypothesized that changing the molecular structure via simplification of the surfactant headgroup and elongation to a single chainlength (making them more structurally similar to typical surfactants) would increase surfactancy.

One of the production strategies previously disclosed involved a short-term, semi-synthetic fermentation approach, in which commercial rhamnolipids were leveraged as the feedstock and hydrolyzed to produce simplified mono-lipid glycolipid congener structures.

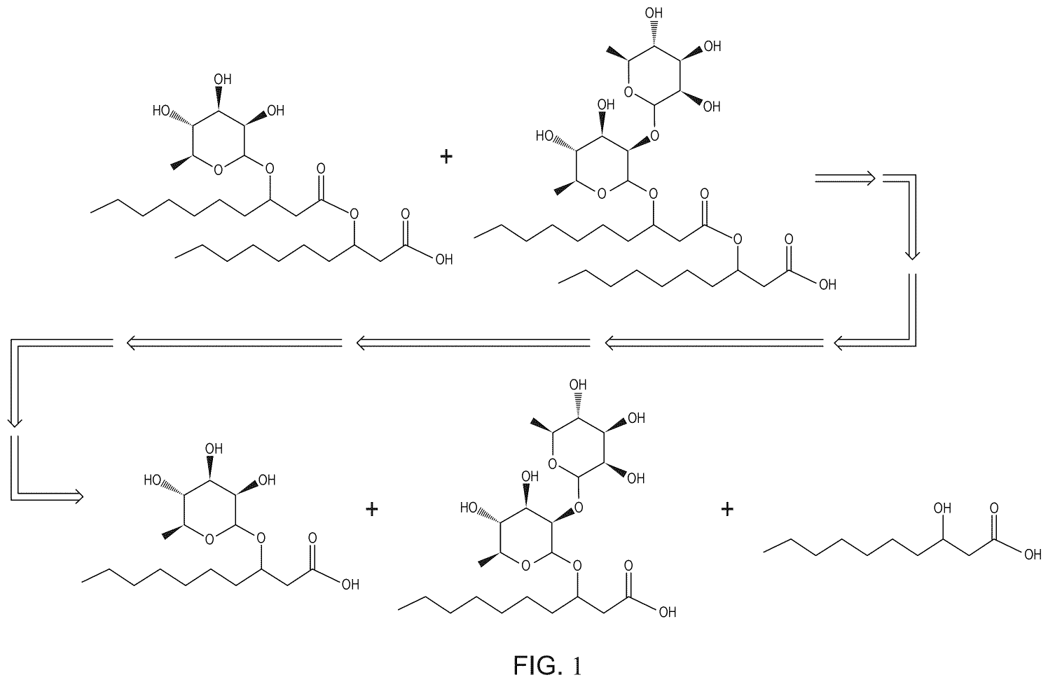

Commercial Rheance® One rhamnolipids (made by Evonik Industries AG, Essen, Germany) were used to demonstrate nearly complete hydrolysis from a monorhamnodilipid plus dirhamnodilipid species to a monorhamnomonolipid plus dirhamnomonolipid species, as exemplified in FIG. 1.

Similar hydrolysis can be done on other commercially available rhamnolipids, such as those sold by BioReNuva, Austin TX, USA, and by Wanhua Chemical Group Co., Ltd., Yantai, China. These hydrolyzed rhamnolipid mixtures offer commercial and consumer benefits. These hydrolyses were all done with a base, such as NaOH.

The present inventors have discovered that performing the hydrolysis of commercial rhamnolipids with a divalent base, or a mixture of divalent and monovalent bases, produces a mixture of monolipid rhamnolipid species. These mixtures may be found to improve a formulated material's surfactancy. In the present invention, a fermentation-based chemical approach is used, in which commercial rhamnolipids are leveraged as the feedstock and chemically hydrolyzed to produce the simplified mono-lipid glycolipid structures.

The present invention involves the treatment of rhamnodilipids with a divalent base under certain conditions of temperature and time, resulting in a rhamnomonolipid hydrolysate. The total result of the hydrolysis comprises rhamnomonolipids and by-products. Through any number of separation techniques, the rhamnomonolipid hydrolysate can be separated from the by-products, and thus the method produces a significant yield of rhamnomonolipids. One advantage of the rhamnomonolipid compositions of the instant invention is their improved color profile, resulting in lighter colors of rhamnomonolipids. Another advantage of the present invention is that the rhamnomonolipid compositions of the instant invention have excellent foaming properties.

Reference within the specification to “embodiment(s)” or the like means that a particular material, feature, structure and/or characteristic described in connection with the embodiment is included in at least one embodiment, optionally a number of embodiments, but it does not mean that all embodiments incorporate the material, feature, structure, and/or characteristic described. Furthermore, materials, features, structures and/or characteristics may be combined in any suitable manner across different embodiments, and materials, features, structures and/or characteristics may be omitted or substituted from what is described. Thus, embodiments and aspects described herein may comprise or be combinable with elements or components of other embodiments and/or aspects despite not being expressly exemplified in combination, unless otherwise stated or an incompatibility is stated.

All ingredient percentages described herein are by weight of the cosmetic composition, unless specifically stated otherwise, and may be designated as “wt %.” All ratios are weight ratios, unless specifically stated otherwise. All ranges are inclusive and combinable. The number of significant digits conveys neither a limitation on the indicated amounts nor on the accuracy of the measurements. All numerical amounts are understood to be modified by the word “about” unless otherwise specifically indicated. Unless otherwise indicated, all measurements are understood to be made at approximately 25° C. and at ambient conditions, where “ambient conditions” means conditions under about 1 atmosphere of pressure and at about 50% relative humidity. All numeric ranges are inclusive of narrower ranges, and delineated upper and lower range limits are interchangeable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods. As used in the description and the appended claims, the singular forms “a,” “an,” and “the” are intended to include the plural forms as well, unless the context clearly indicates otherwise.

### Definitions

When used in the context of a chemical group: “hydrogen” means —H; “hydroxy” means —OH; “oxo” means ═O; “carbonyl” means —C(═O)—; “carboxy” and “carboxylate” mean —C(═O)OH (also written as —COOH or —CO₂H) or a deprotonated form thereof; “amino” means —NH₂; “hydroxyamino” means —NHOH; “nitro” means —NO₂; “imino” means ═NH; “amine oxide” means N⁺O⁻ where N has three covalent bonds to atoms other than O; “hydroxamic” or “hydroxamate” means —C(O)NHOH or a deprotonated form thereof.

The term “cation” refers to an atom, molecule, or a chemical group with a net positive charge including single and multiply charged species. Cations can be individual atoms such as metals, non-limiting examples include Na⁺ or Ca⁺², individual molecules, non-limiting examples include (CH₃)₄N⁺, or a chemical group, non-limiting examples include —N(CH₃)₃⁺. The term “amine cation” refers to a particular molecular cation, of the form NR₄⁺ where the four substituting R moieties can be independently selected from H and alkyl, non-limiting examples include NH₄⁺ (ammonium), CH₃NH₃⁺ (methylammonium), CH₃CH₂NH₃⁺ (ethylammonium), (CH₃)₂NH₂⁺ (dimethylammonium), (CH₃)₃NH⁺ (trimethyl ammonium), and (CH₃)₄N⁺ (tetramethylammonium).

The term “anion” refers to an atom, molecule, or chemical group with a net negative charge including single and multiply charged species. Anions can be individual atoms, for example but not limited to halides F⁻, Cl⁻, Br⁻, individual molecules, non-limiting examples include CO₃⁻², H₂PO₄⁻, HPO₄⁻², PO₄⁻³, HSO₄, SO₄⁻², or a chemical group, non-limiting examples include sulfate, phosphate, sulfonate, phosphonate, phosphinate, sulfonate, mercapto, carboxylate, amine oxide, hydroxamate and hydroxyl amino. Deprotonated forms of previously defined chemical groups are considered anionic groups if the removal of the proton results in a net negative charge. In solutions, chemical groups are capable of losing a proton and become anionic as a function of pH according to the Henderson-Hasselbach equation (pH=pKa+log₁₀([A⁻]/[HA]; where [HA] is the molar concentration of an undissociated acid and [A⁻] is the molar concentration of this acid's conjugate base). When the pH of the solution equals the pKa value of functional group, 50% of the functional group will be anionic, while the remaining 50% will have a proton. Typically, a functional group in solution can be considered anionic if the pH is at or above the pKa of the functional group.

The term “salt” or “salts” refers to the charge neutral combination of one or more anions and cations. For example, when R is denoted as a salt for the carboxylate group, —COOR, it is understood that the carboxylate (—COO—) is an anion with a negative charge-1, and that the R is a cation with a positive charge of +1 to form a charge neutral entity with one anion of charge −1, or R is a cation with a positive charge of +2 to form a charge neutral entity with two anions both of −1 charge.

The term “saturated” as used herein means the chemical compound or group so modified has no carbon-carbon double and no carbon-carbon triple bonds, except as noted below. In the case of substituted versions of saturated chemical groups, one or more carbon oxygen double bond or a carbon nitrogen double bond may be present. When such a bond is present, then carbon-carbon double bonds that may occur as part of keto-enol tautomerism or imine/enamine tautomerism are not precluded.

The term “aliphatic” when used without the “substituted” modifier signifies that the chemical compound/group so modified is an acyclic or cyclic, but non-aromatic hydrocarbon chemical compound or group. In aliphatic chemical compounds/groups, the carbon atoms can be joined together in straight chains, branched chains, or non-aromatic rings (alicyclic). Aliphatic chemical compounds/groups can be saturated, that is joined by single bonds (alkanes/alkyl), or unsaturated, with one or more double bonds (alkenes/alkenyl), or with one or more triple bonds (alkynes/alkynyl).

The term “alkyl” when used without the “substituted” modifier refers to a monovalent saturated aliphatic group with a carbon atom as the point of attachment, a linear or branched, cyclo, cyclic, or acyclic structure, and no atoms other than carbon and hydrogen. Thus, as used herein cycloalkyl is a subset of alkyl, with the carbon atom that forms the point of attachment also being a member of one or more non-aromatic ring structures wherein the cycloalkyl group consists of no atoms other than carbon and hydrogen. As used herein, the term does not preclude the presence of one or more alkyl groups (carbon number limitation permitting) attached to the ring or ring system. The groups —CH₃ (Me), —CH₂CH₃ (Et), —CH₂CH₂CH₃ (n-Pr or propyl), —CH(CH₃)₂ (i-Pr, ‘Pr, or isopropyl), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tertbutyl, t-butyl, t-Bu, or tBu), —CH₂C(CH₃)₃ (neo-pentyl), cyclobutyl, cyclopentyl, cyclohexyl, and cyclohexylmethyl are non-limiting examples of alkyl groups. The term “alkanediyl” when used without the “substituted” modifier refers to a divalent saturated aliphatic group, with one or two saturated carbon atom(s) as the point(s) of attachment, a linear or branched, cyclo, cyclic or acyclic structure, no carbon-carbon double or triple bonds, and no atoms other than carbon and hydrogen. The groups, —CH₂-(methylene), —CH₂CH₂—, —CH₂C(CH₃)₂CH₂—, and —CH₂CH₂CH₂— are non-limiting examples of alkanediyl groups. The term “alkylidene” when used without the “substituted” modifier refers to the divalent group ═CRR′ in which R and R′ are independently hydrogen, alkyl, or R and R′ are taken together to represent an alkanediyl having at least two carbon atoms. Non-limiting examples of alkylidene groups include: ═CH₂, ═CH(CH₂CH₃), and ═C(CH₃)₂. An “alkane” refers to the compound H—R, wherein R is alkyl as this term is defined above.

When any of these terms is used with the “substituted” modifier one or more hydrogen atom has been independently replaced by —OH, —F, —Cl, —Br, —I, —NH₂,—NO₂, —CO₂H, —CO₂CH₃, —CN, —SH, —OCH₃, —OCH₂CH₃, —C(O)CH₃, —NHCH₃, —NHCH₂CH₃, —N(CH₃)₂, —C(O)NH₂, —OC(O)CH₃, —S(O)₂NH₂, —P(O)(OH)₂, —P(O)(OH) OP(O)(OH)₂, —OP(O)(OH)₂, —OP(O)(OH) OP(O)(OH)₂, —S(O)₂(OH), or —OS(O)₂(OH). The following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N (CH₃)₂, —CH₂CH₂Cl, —CH₂P(O)(OH)₂, —CH₂P(O)(OH) OP(O)(OH)₂, —CH₂S (O)₂(OH), and —CH₂OS(O)₂(OH). The term “haloalkyl” is a subset of substituted alkyl, in which one or more hydrogen atoms has been substituted with a halo group and no other atoms aside from carbon, hydrogen and halogen are present. The group, —CH₂Cl is a non-limiting example of a haloalkyl. The term “fluoroalkyl” is a subset of substituted alkyl, in which one or more hydrogen has been substituted with a fluoro group and no other atoms aside from carbon, hydrogen and fluorine are present. The groups, —CH₂F, —CF₃, and —CH₂CF₃ are non-limiting examples of fluoroalkyl groups.

The term “alkenyl” when used without the “substituted” modifier refers to a monovalent unsaturated aliphatic group with a carbon atom as the point of attachment, a linear or branched, cyclo, cyclic or acyclic structure, at least one nonaromatic carbon-carbon double bond, no carbon-carbon triple bonds, and no atoms other than carbon and hydrogen. Non-limiting examples of alkenyl groups include: —CH═CH₂ (vinyl), —C(CH₃)═CH₂ (methyl-vinyl), —CH═CHCH₂CH₃, —CH═CHCH₂CH₃, —CH₂CH═CH₂ (allyl), —CH₂CH═CHCH₃, and —CH═CHCH═CH₂. The term “alkenediyl” when used without the “substituted” modifier refers to a divalent unsaturated aliphatic group, with two carbon atoms as points of attachment, a linear or branched, cyclo, cyclic or acyclic structure, at least one nonaromatic carbon-carbon double bond, no carbon-carbon triple bonds, and no atoms other than carbon and hydrogen. The groups, >C═CH₂ (vinylidine), —CH═CH—, —CH═C(CH₃)CH₂—, and —CH═CHCH₂—, are non-limiting examples of alkenediyl groups. It is noted that while the alkenediyl group is aliphatic, once connected at both ends, this group is not precluded from forming part of an aromatic structure. The terms “alkene” or “olefin” are synonymous and refer to a compound having the formula H—R, wherein R is alkenyl as this term is defined above.

The term “aryl” when used without the “substituted” modifier refers to a functional group derived from a simple aromatic ring compound where the point of attachment is a carbon atom on the aromatic ring. An aromatic ring is a hydrocarbon that has a cyclic structure and a delocalized electron system. An aryl group is formed by removing one hydrogen atom from the ring. The name of the aryl group is based on the name of the aromatic ring with the -yl suffix, such as phenyl, naphthyl, indolyl, etc. Aryl groups may be substituted with alkyl and/or heteroalkyl chains and may have one or more heteroatoms within the aryl ring.

The term “arylalkyl” refers to an aryl group attached to an alkanediyl group where the point of attachment is on the alkanediyl group.

Tauryl/taurate is defined as aminoethyl sulphonyl.

Hetero is defined as an atom other than carbon, including but not exclusive to, nitrogen (N), oxygen (O), or sulfur(S). The heteroatom may be attached in a linear or branched alkyl chain or also may be attached to a non-aromatic or aromatic ring, either as part of the ring, or adjacent to it as a substituent. There may be more than one heteroatom in the alkyl chain or ring.

Unsaturated is defined as a hydrocarbon chain possessing, but not limited, at least one carbon-carbon double bond (C═C). The unsaturated chain may possess one double bond (alkenyl), two double bonds (dienyl), multiple double bonds (polyenyl), and/or a carbon-carbon triple bond (acetylenic). The unsaturated bonds may be adjacent (conjugated) relative to each other or separated by additional carbon atoms in the chain.

A “monomer molecule” is defined by the International Union of Pure and Applied Chemistry (IUPAC) as “A molecule which can undergo polymerization thereby contributing constitutional units to the essential structure of a macromolecule.” A polymer is a macromolecule.

“Treat” or “treating” as used in reference to a composition, means to add or apply a material to the composition.

“About” modifies a particular value by referring to a range of plus or minus 20% or less of the stated value (e.g., plus or minus 15% or less, 10% or less, 5% or less, or even 1% or less). “Apply” or “application,” as used in reference to a composition, means to apply or spread the composition onto a human keratinous surface such as the skin or hair.

“Charge density” (“CD”) means the ratio of positive charges on a polymer to the molecular weight of the polymer.

“Personal care composition” is meant a product, which in the ordinary course of usage is applied to or contacted with a body surface to provide a beneficial effect. Body surface includes skin, for example dermal or mucosal; body surface also includes structures associated with the body surface for example hair, teeth, or nails. Examples of personal care compositions include a product applied to a human body for improving appearance, cleansing, and odor control or general aesthetics. Non-limiting examples of personal care compositions include oral care compositions, such as, dentifrice, mouth rinse, mousse, foam, mouth spray, lozenge, chewable tablet, chewing gum, tooth whitening strips, floss and floss coatings, breath freshening dissolvable strips, denture care product, denture adhesive product; after shave gels and creams, pre-shave preparations, shaving gels, creams, or foams, moisturizers and lotions; cough and cold compositions, gels, gel caps, and throat sprays; leave-on skin lotions and creams, shampoos, body washes, body rubs, such as Vicks VapoRub; hair conditioners, hair dyeing and bleaching compositions, mousses, shower gels, bar soaps, antiperspirants, deodorants, depilatories, lipsticks, foundations, mascara, sunless tanners and sunscreen lotions; feminine care compositions, such as lotions and lotion compositions directed towards absorbent articles; baby care compositions directed towards absorbent or disposable articles; and oral cleaning compositions for animals, such as dogs and cats.

“Pharmaceutical product” refers to a drug used to diagnose, cure, treat, or prevent disease.

“Solid crystalline” refers to the crystalline structure of the lamellar or vesicular phase at ambient temperatures caused by the phase being below its melt transition temperature. For example, the melt transition temperature of the lamellar or vesicular phase may be about 30° C. or more (i.e., slightly above about room temperature). The melt transition temperature can be measured through differential scanning calorimetry, which is conventional measurement method known to those skilled in the art.

“Substantially free of” means a composition or ingredient comprises less than 3% of a subject material, by weight of the composition or ingredient (e.g., less than 2%, less than 1% or even less than 0.5%). “Free of” means a composition or ingredient contains 0% of a subject material.

“Sulfated surfactants” means surfactants that contain a sulfate moiety. Some non-limiting examples of sulfated surfactants are sodium lauryl sulfate, sodium laureth sulfate, ammonium lauryl sulfate, and ammonium laureth sulfate. “Sulfate-free surfactant” refers to a surfactant that has no sulfate moieties.

“Separation” is defined as a method that converts a mixture or solution of chemical substances into two or more distinct product mixtures. At least one of the results of the separation is a supplement in one or more of the source mixture's constituents. In some cases, a separation process may fully divide the mixture into its pure constituents. Separation may include, but is not limited to, filtration, nanofiltration, centrifugation, chromatography and crystallization.

“Hydrolysate” in general is a product of hydrolysis. Herein, rhamnomonolipid hydrolysate refers to the rhamnomonolipid product made by the treatment of a rhamnodilipid material with a basic reagent in a pH range of 8-14; total hydrolysate refers to the rhamnomonolipid hydrolysate plus by-products.

“Cx” may be independently defined as or selected from an alkyl, aryl, heteroalkyl, heteroaryl, unsaturated alkyl, and unsaturated heteroalkyl, wherein each Cx independently has a carbon chain length from 4 to 22, or in some embodiments from 5 to 13, or in some embodiments the carbon chain length may be 10 or may be 12.

A “divalent cation” is defined as a positively charged ion that has lost two electrons and has a valence of 2+. This means that it can form two chemical bonds with anions, which are negatively charged ions. Some examples of divalent cations are magnesium (Mg 2+), calcium (Ca 2+), and iron (Fe 2+).

A “beta-hydroxy acid” is defined as a molecule that possesses a hydroxyl group (OH) two atoms adjacent from a carboxylic acid group (CO₂H).

A “basic reagent” is defined as a chemical that raises pH level above 7 when added to water. Basic reagents include NaOH, Ca(OH)₂, LiOH, KOH, Mg(OH)₂ and Fe(OH)₂ as well as many others. Basic reagents may be inorganic or organic in nature.

A “decolorizing agent” is defined as a chemical substance and/or particle used to remove or reduce color from a solution or material. Decolorizing agents are commonly used in various industries and applications where the presence of color is undesirable, such as in the purification of liquids, refining processes, and wastewater treatment.

“Nanofiltration” is defined as a membrane filtration process that uses nanometer sized pores through which particles smaller than about 1-10 nanometers pass through the membrane. Nanofiltration membranes have pore sizes of about 1-10 nanometers, smaller than those used in microfiltration and ultrafiltration, but a slightly bigger than those in reverse osmosis. Membranes used are predominantly polymer thin films. It is used to soften, disinfect, and remove impurities from water, and to purify or separate chemicals such as pharmaceuticals.

“Filtration” is defined as a physical separation process that separates solid matter and fluid from a mixture using a filter medium that has a complex structure through which only the fluid can pass. Solid particles that cannot pass through the filter medium are described as oversize and the fluid that passes through is called the filtrate. Oversize particles may form a filter cake on top of the filter and may also block the filter lattice, preventing the fluid phase from crossing the filter, known as blinding. The size of the largest particles that can successfully pass through a filter is called the effective pore size of that filter. The separation of solid and fluid is imperfect; solids will be contaminated with some fluid and filtrate will contain fine particles (depending on the pore size, filter thickness and biological activity). Filtration occurs both in nature and in engineered systems; there are biological, geological, and industrial forms.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### Rhamnolipid Surfactant

Rhamnose (Rha, Rham), formula illustrated below, is a naturally occurring deoxy sugar. It can be classified as either a methyl-pentose or a 6-deoxy-hexose.

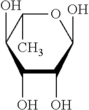

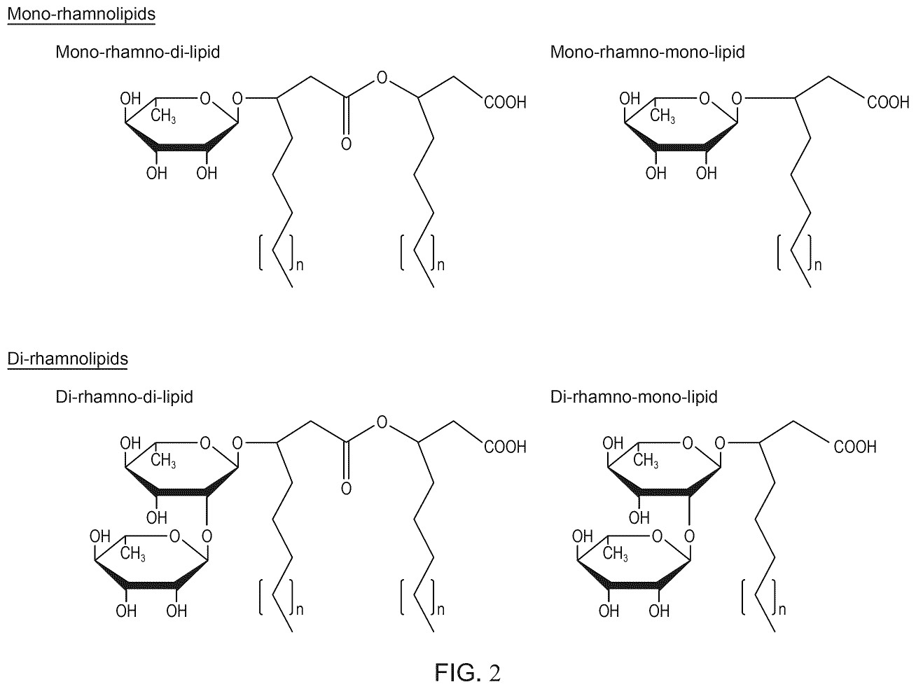

Rhamnolipids are a class of glycolipid that may be used as bacterial surfactants. They have a glycosyl head group, in this case a rhamnose moiety, and an acid fatty tail (lipid). There are two main classes of rhamnolipids: mono-rhamnolipids and di-rhamnolipids, which consist of one or two rhamnose groups respectively. And then mono-rhamnolipids and di-rhamnolipids may each have either one or two lipids, creating, for example, the molecules shown in FIG. 2.

The compositions described herein include one or more rhamnolipid biosurfactants. The rhamnolipid surfactants herein may be produced by microorganisms (e.g., Pseudomonas aeruginosa, Pseudomonas putida, Pseudomonas chlororaphis). The rhamnolipid surfactant(s) may provide a cleaning benefit due to their amphiphilic nature, which allows the surfactants to break up, and form micelles around, oil and other contaminants. The “entrapped” contaminant can then be rinsed off more easily with water. A description of various types of rhamnolipids is disclosed in EP2410039. Methods of making, extracting, and blending naturally produced rhamnolipids are known in the art.

### Divalent Base

The basic divalent bases generally suitable for use in the hydrolysis of commercial rhamnolipids are those selected from the group consisting of alkali earth metals, such as magnesium, calcium, strontium, barium: alkali earth metal salts, such as calcium hydroxide, calcium bicarbonate, calcium carbonate, magnesium oxide, magnesium hydroxide, strontium hydroxide, or barium hydroxide.

In another particularly preferred embodiment of the present invention, the basic catalyst used in the reaction is calcium hydroxide, or mixtures of calcium hydroxide with monovalent bases such as sodium hydroxide or potassium hydroxide. It has been found that when these specific compounds are used as bases, light colored mixture of monolipid rhamnolipid species are obtained when compared to essentially identical reactions carried out using only monovalent bases, such as sodium hydroxide or potassium hydroxide. Calcium hydroxide is the most preferred divalent base for use herein.

The level of divalent base is used as low as possible, typically from about 0.5 to about 1.5, preferably from about 0.9 to about 1.1, moles of catalyst per mole of commercial or starting rhamnolipids (rhamnodilipids).

The present inventors have discovered that performing the hydrolysis of commercial rhamnolipids with a divalent base, or a mixture of divalent and monovalent bases, produces a mixture of monolipid rhamnolipid species. These mixtures may be found to improve a formulated material's surfactancy. In the present invention, a fermentation-based chemical approach is used, in which commercial rhamnolipids are leveraged as the feedstock and chemically hydrolyzed to produce the simplified mono-lipid glycolipid structures. Furthermore, by judicious choice of the chemical used in the hydrolysis, by-products removal can occur by separation after hydrolysis is complete.

For example, commercially available Rheance One rhamnolipids, sold by Evonik, were nearly completely hydrolyzed from a monorhamnodilipid and dirhamnodilipid species to a monorhamnomonolipid and dirhamnomonolipid species, as shown in FIG. 1.

Rhamnolipids can be made both chemically and biologically. Chemical synthesis of mono-rhamno-mono-lipids have been described previously, via both de-novo synthesis (ex: Synthesis and Characterization of Glycoside Non-Ionic Surfactants; Nagila, M. P. S.; Ricardo, A. G.; Price*, C.; Polymer and Science, Engineering 1996, 36 (2), p. 182-1987.; US2023114778A1; US20230132332A1; US20230278976A1; US20240173456A1) and/or via hydrolysis of other rhamnolipids (ex: Rhamnolipids Sustain Unchanged Surface Activities during Decomposition in Alkaline Solutions.; Shuai Kong, Chong Shen, Yizeng Li, and Qin Meng*; ACS Omega 2021, 6, 15750-15755. There remains a need to control product cost and purity profile with separation techniques amenable to large scale production of synthetic rhamnolipids. It has been reported that large scale purification of di-rhamnolipids from fermentation has been achieved using CaCl₂) (CN111233948).

One step of the present invention involves treating the rhamnodilipids with a composition comprising a divalent base at a temperature sufficient to cause hydrolysis. Such a step may be done at a temperature from about 25° C. to about 100° C. In addition, such a step may be done for a time period anywhere from about 30 minutes to about 100 hours.

The present inventors have found that synthesis of modified rhamnolipids, such as the rhamnomonolipid class, is attainable on scale by using a divalent base during the hydrolysis of commercial dirhamnodilipds. The by-products of the reaction are those compounds derived from the resulting hydrolysis reaction, and may include β-hydroxy acids, which may be as insoluble divalent salts. Further by-products may include a,b-unsaturated acids.

Separation of the rhamnomonolipid hydrolysate from the by-products may be done. Appropriate methods to separate the rhamnomonolipid hydrolysate from any by-product may include, without being limited to, filtration, centrifugation, precipitation, and combinations thereof. Types of filtration may include nano-filtration.

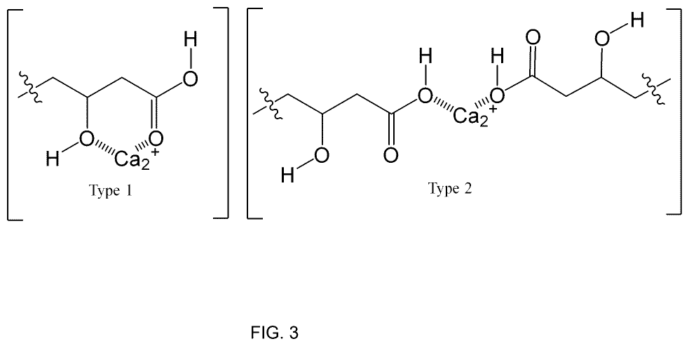

As an example, divalent cations have the potential to chelate in a variety of different manners. In the case of rhamnomonolipids, and by-product acid divalent salts, it could be envisioned that two potential chelation modes are applicable, depending upon molecule concentration and solvents effects, as shown in FIG. 3.

In a Type 1 mechanism in FIG. 3, the cation may chelate intramolecularly between the carboxylate group and the adjacent b-hydroxyl group in a 6-membered ring transition state manner, within the same molecule. This may be particularly relevant at pH where the acid moiety is protonated. In a Type 2 chelation event in FIG. 3, the metal cation may form an intermolecular bridge between two molecules of b-hydroxy acid, similar to studies reported for Atorvastatin Calcium salt (Ryan L.; et. al; Powder Diffraction, 35 (2), June 2020, p. 136-143). Based on chelation structure, solubility properties of either species could be significant. These structural features can potentially be utilized in the selective separative removal of by-product b-hydroxy acid side-chains when dirhamnodilipids are treated with specific bases that possess divalent cations.

The degree of hydrolysis of the rhamnodilipids may be from about 30% to about 100%, and in some embodiments from about 50% to about 70%.

In some embodiments, the total hydrolysate may comprise less than about 10% of by-products or less than about 5% of by-products.

In some embodiments, the rhamnodilipids are provided in a solvent, and the method may further comprise the step of removing the solvent from the rhamnomonolipid hydrolysate.

It has been discovered that rhamnolipid hydrolysate resulting from divalent base hydrolysis can be modified in color and composition by the choice of divalent base used in the chemical hydrolysis, and by separation. Acid by-products may be simply removed by standard separation techniques to provide desired rhamnolipid by using divalent bases in the hydrolysis. By-product removal can be accomplished by filtration, centrifugation, or combination of both. This has the effect that the rhamnomonolipid hydrolysate product has an improved color.

In some embodiments, the method may comprise the steps of separating the rhamnomonolipid hydrolysate from the by-products and then adjusting the pH of the rhamnomonolipid hydrolysate to be at least 2.

Before separation of the rhamnolipids and by-products, the rhamnolipid compositions comprise primarily (a), and (b), with (c) being a by-product (for example, divalent salt of beta hydroxy fatty acid). Optionally, (c) may be removed or separated from (a) and (b). The compositions of (a), (b), and (c) are disclosed below:

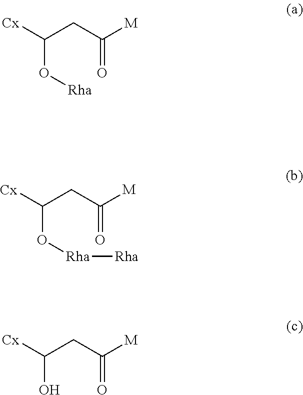

wherein Rha is rhamnose;
wherein each Cx is independently selected from an alkyl, aryl, heteroalkyl, heteroaryl, unsaturated alkenyl, and unsaturated heteroalkenyl;
wherein each Cx independently has a carbon chain length from 4 to 22;
wherein each M is independently selected from O−X+ wherein X+ is a divalent cation selected from Ca2+, Mg2+Fe2+, Ba2+, and Sr2+, and all possible stereoisomers thereof.

Use of the divalent base allows the non-rhamnolipid compositions to easily be separated from the rhamnolipid compositions, as the by-products can typically be filtered or centrifuged out. The remaining composition is then almost entirely (a) and (b), which is the desired rhamnomonolipids. After separation of the by-products (c) from the rhamnomonolipids (a) and (b), the remaining composition may be at least about 80%, at least about 90%, at least about 95%, at least about 98%, or at least about 99% rhamnomonolipids.

Cx may be independently defined as or selected from an alkyl, aryl, heteroalkyl, heteroaryl, unsaturated alkyl, and unsaturated heteroalkyl, wherein each Cx independently has a carbon chain length from 4 to 22; more specifically, Cx can be the C-3 carbon (C5-C13) chain pendant to the glyceride carboxy terminus as shown in Table 1.

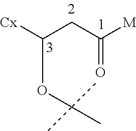

TABLE 1

| Cx | Name |
|---|---|
| 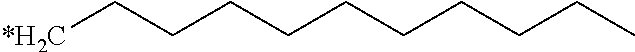 | Undecyl (C11) |
| 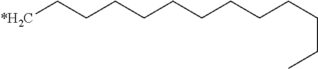 | Tridecyl (C13) |
| 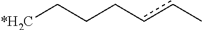 | Heptenyl (C7:1) |
| 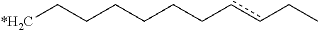 | Undecenyl (C11:1) |
| 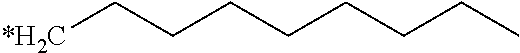 | Nonyl (C9) |
| 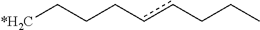 | Nonenyl (C9:1) |
| 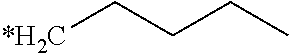 | Pentyl (C5) |
| 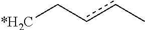 | Pentenyl (C5:1) |
| 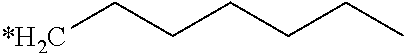 | Heptyl (C7) |
| 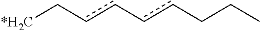 | Nonadienyl (9:2) |

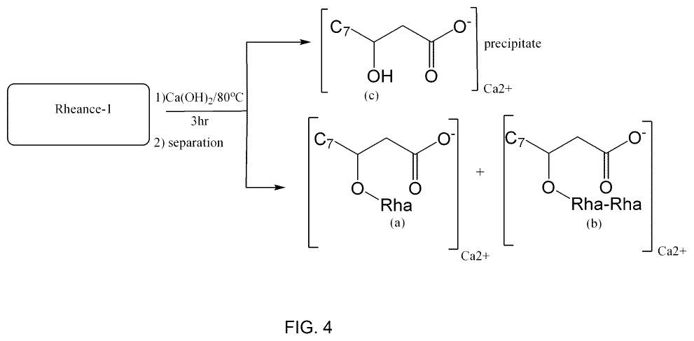

Table 2 shows the starting material and major products of Inventive Example 1, the synthesis and separation of calcium hydrolysate and b-hydroxy decanoic acid calcium salt (schematic shown in FIG. 4).

### Example 1

In a 3 L, 3-neck round-bottomed flask equipped with reflux condenser with N₂ inlet regulator, Caframo RZR-2000 Overhead Stirrer, and J-Kem Scientific Temperature Controller with probe was charged with a 400 grams weight solution of Rheance® One, (Evonik, 50% weight in H₂O, 200 g, Mw˜650, 0.307 mol), followed by 750 mL water, and Ca(OH)₂ powder (Sigma-Aldrich, CAS 1305-62-0, Mw=74.09, 50 grams, 0.676 mol). The reaction was heated to 80° C. and stirred for 3 hours. The reaction remained heterogeneous the entire time, and viscosity increased significantly. The reaction was cooled to room temperature, and the solids filtered through a glass-sintered fritted Buchner funnel (Pyrex®, 3000 mL, C-frit). The mother liquor was then directly lyophilized to remove water. (Virtis Model #50L Virtual XL-70, 130 mTorr, 72 hrs, 0° C.-room temperature shelf temperature over 24 h, then 48 hat RT). The result was a white powder, C10-Rha-Rha (b) wherein M=O—Ca++ (122.5 grams, wet weight, MS m/z ESI, 479). The filtered material was dried under vacuum (5-10 mmHg) for 72 hours to give a white solid, product (c) wherein M=O—Ca++ and Cx=heptyl (110 grams, wet weight, MS m/z ESI, 187). Samples may be neutralized with the appropriate acid to provide the free acid form.

The following Table 2 shows that complete conversion occurs of the starting rhamnodilipid composition. Peak differentiation in the 13C NMR spectrum of each of the products clearly showed a difference in chemical shift in the carbonyl region (C═O) as hydrolysis proceeded, starting from Rheance One (R) (C═O resonances at 175.2, 171.4), to the hydrolyzed monolipid carbonyl peak (C═O) at 179.2, to the 3-hydroxy decanoic acid product at 174.2. These 1C NMR resonances are diagnostic for monitoring the complete conversion of dilipid-Rha-Rha to monolipid-Rha-Rha and by-product 3-hydroxy decanoic acid.

TABLE 2
Ca2+ Rhamnolipid Hydrolysate Products; 13C NMR Major Peak Assignments (d4-MeOH/ppm)

| | Carbonyl | |
|---|---|---|
| | Region | |
| Structure | (C═O) | Other Resonances |
| 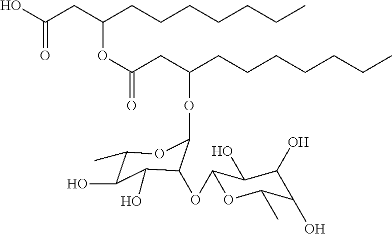 [SMILES: CCCCCCCC(CC(=O)O)OC(=O)CC(CCCCCCC)OC1OC(C)C(O)C(O)C1OC1OC(C)C(O)C(O)C1O] | 175.2, 171.4 | 129.1, 127.7, 102.8, 97.0, 79.2, 73.2, 73.1, 72.8, 72.5, 72.0, 70.8, 70.5, 70.3, 68.8, 68.7, 48.3, 42.6, 40.5, 33.9, 32.4, 31.5, 29.4, 29.1, 28.9, 28.7, 25.2, 24.5, 24.3, 16.6, 13.0. |
| Chemical Formula: C₃₂H₅₈O₁₃ | | |
| Rheance® One | | |
| 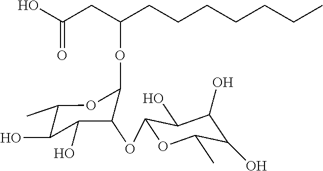 [SMILES: CCCCCCCC(CC(=O)O)OC1OC(C)C(O)C(O)C1OC1OC(C)C(O)C(O)C1O] | 179.2 | 128.9, 127.3, 102.7, 97.1, 79.1, 74.8, 73.1, 72.5, 70.9, 70.7, 68.8, 68.5, 47.0. 32.9, 31.6, 29.6, 28.0, 24.5, 22.3, 16.7, 13.0 |
| Chemical Formula: C₂₂H₄₆O₁₁ | | |
| 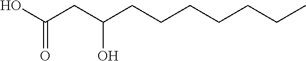 | 174.2 | 67.9, 47.6, 36.7, 31.6, 29.3, 29.0, 25.2, 22.3, 13.0. |
| Chemical Formula: C₁₀H₂₀O₃ | | |

All NMR (nuclear magnetic resonance spectroscopy) spectrum data were obtained on a Bruker NanoBay 300 mHz NMR with AvanceIII processor and SampleXpress sample changer using deuterated methanol (CD₃OD) as solvent. Mass spectrum were performed on a Thermoscientific Vanquish mass spectrometer using via negative mode (ES-) electrospray ionization.

For Example 1, the by-product was separated out and the remaining composition tested, where it was found that the level of beta hydroxy acid (C10 hydroxy acid) was about 1.35%. This means that the inventive method can produce a composition that is at least 98% rhamnomonolipids.

The absolute quantification of C10 hydroxy acid in the inventive Example 1 was done by UPLC-MS/MS chromatography consisted of an ACQUITY BEH C18 Vanguard precolumn (130 Å, 1.7 μm, 2.1 mm×5 mm, Waters, Milford, MA, USA) followed by an ACQUITY UPLC BEH C18 Column (130 Å, 1.7 μm, 2.1 mm×100 mm, Waters, Milford, MA, USA). The LC gradient was carried out on Sciex ExionLC system (Sciex, Framingham, MA, USA) from 5 to 95% B (Acetonitrile) over 10.5 min, for a total run time of 15 min. Mobile Phase A was 4 mM Ammonium Acetate in Water, and the flow rate was set to 0.4 mL/min. The column temperature was kept at 45° C. in the column compartment.

Detection and quantitation of C10 hydroxy acid were performed tandem mass spectrometry on a Triple Quad 6500+ system (Sciex, Framingham, MA, USA) operating under multiple reaction monitoring (MRM) and negative electrospray ionization (ESI) mode (C10 hydroxy acid: 187.1→59.0).

All data processing was performed using the Skyline Software, Version 23.1.0.268 (MacCoss Lab, University of Washington, Seattle, WA, USA). The concentration of C10 hydroxy acid in the study sample was determined from its corresponding peak area by interpolation from a weighted (1/x2) quadratic regression curve of calibration standards. The nominal range of quantitation was 250 ng/mL to 22000 ng/mL.

Optionally, the rhamnomonolipids hydrolysate product obtained can be treated with a decoloring agent, such as an aqueous solution of hydrogen peroxide, preferably by applying a net/active amount of hydrogen peroxide of 0.01 to 1.0, preferably from 0.05 to 0.25 for 30 min to 500 min at 20° C. to 50° C.

In addition, the B-hydroxy acid side product resulting from the hydrolysis reaction can be recovered in high purity and yield and commercially used.

### EXAMPLES/COMBINATIONS

A. A method of making a rhamnomonolipid hydrolysate by the following steps:
   a. providing rhamnodilipids;
   b. treating the rhamnodilipids with a composition comprising a divalent base at a temperature sufficient to cause hydrolysis;
   c. producing a rhamnomonolipid hydrolysate and by-products; and
   d. optionally separating the rhamnomonolipid hydrolysate from the by-products.
B. The method of Paragraph A, wherein step (b) is done at a temperature from about 25° C. to about 100° C.
C. The method of Paragraph A or B, wherein step (b) is done for a time period from about 30 minutes to about 100 hours.
D. The method of any one of Paragraph A-C, wherein the divalent base is selected from calcium hydroxide, calcium bicarbonate, calcium carbonate, magnesium oxide, magnesium hydroxide, strontium hydroxide, barium hydroxide, and combinations thereof.
E. The method of any one of Paragraph A-D, wherein the divalent base is calcium hydroxide.
F. The method of any one of Paragraph A-E, wherein the composition comprising a divalent base is a mixture of mono- and divalent bases.
G. The method of any one of Paragraph A-F, wherein the monovalent base is selected from sodium hydroxide, potassium hydroxide, and combinations thereof.
H. The method of any one of Paragraph A-G, wherein the composition comprising a divalent base is 100% a divalent base.
I. The method of any one of Paragraph A-H, wherein the composition comprising a divalent base is present in an amount from about 0.5 to about 1.5 moles per mole of rhamnodilipids.
J. The method of any one of Paragraph A-I, wherein the composition comprising a divalent base is present in an amount from about 0.9 to about 1.1 moles per mole of rhamnodilipids.
K. The method of any one of Paragraph A-J, wherein the degree of hydrolysis is from about 30% to about 100%.
L. The method of any one of Paragraph A-K, wherein the degree of hydrolysis is from about 50% to about 70%.
M. The method of any one of Paragraph A-L, wherein the rhamnodilipids are provided in a solvent, and wherein the method further comprises the step of removing the solvent from the rhamnomonolipid hydrolysate.
N. The method of any one of Paragraph A-M, further comprising the step of treating the rhamnomonolipid hydrolysate with one or more decolorizing agents.
O. The method of any one of Paragraph A-N, wherein the decolorizing agent comprises hydrogen peroxide.
P. The method of any one of Paragraph A-O, wherein the separation of the rhamnomonolipid hydrolysate from the by-products is done by a process selected from filtration, precipitation, centrifugation, and combinations thereof.
Q. The method of any one of Paragraph A-P, wherein the by-products comprise a divalent salt, and the method further comprises the step of separating the rhamnomonolipid hydrolysate from the divalent salt by a process selected from precipitation, filtration, centrifugation, and combinations thereof.
R. The method of any one of Paragraph A-Q, wherein the by-products comprise b-hydroxy acid and the method further comprises the step of separating the b-hydroxy acid from the rhamnomonolipid hydrolysate via precipitation and filtration.
S. The method of any one of Paragraph A-R, wherein the by-products comprise a,b-unsaturated acids and the method further comprises the step of separating the a,b-unsaturated acids from the rhamnomonolipid hydrolysate via precipitation and filtration.
T. The method of any one of Paragraph A-S, wherein the by-products comprise b-hydroxy acid and the method further comprises the step of separating the b-hydroxy acid from the rhamnomonolipid hydrolysate via nano-filtration.
U. The method of any one of Paragraph A-T, wherein the by-products comprise a,b-unsaturated acids and the method further comprises the step of removing the a,b-unsaturated acids from the rhamnomonolipid hydrolysate via nano-filtration.
V. The method of any one of Paragraph A-U, wherein the method further comprises the steps of separating the rhamnomonolipid hydrolysate from the by-products and then adjusting the pH of the rhamnomonolipid hydrolysate to be at least 2.
W. The method of any one of Paragraph A-V, wherein the total hydrolysate comprises less than about 10% of by-products.
X. The method of any one of Paragraph A-W, wherein the total hydrolysate comprises less than about 5% of by-products.
Y. A personal care product comprising the rhamnomonolipid hydrolysate produced in any one of Paragraph A-X.
Z. A pharmaceutical product comprising the rhamnomonolipid hydrolysate produced in any one of Paragraph A-Y.
AA. The use of the rhamnomonolipid hydrolysate made in any one of Paragraph A-Z in a personal care product and/or a pharmaceutical product.

What is claimed is:

1. A method of making a rhamnomonolipid hydrolysate by the following steps:
a. providing rhamnodilipids;
b. treating the rhamnodilipids with a composition comprising a divalent base at a temperature sufficient to cause hydrolysis;
c. producing a rhamnomonolipid hydrolysate and by-products; and
d. optionally separating the rhamnomonolipid hydrolysate from the by-products.

2. The method of claim 1, wherein step (b) is done at a temperature from about 25° C. to about 100° C.

3. The method of claim 1, wherein step (b) is done for a time period from about 30 minutes to about 100 hours.

4. The method of claim 1, wherein the divalent base is selected from calcium hydroxide, calcium bicarbonate, calcium carbonate, magnesium oxide, magnesium hydroxide, strontium hydroxide, barium hydroxide, and combinations thereof.

5. The method of claim 1, wherein the divalent base is calcium hydroxide.

6. The method of claim 1, wherein the composition comprising a divalent base is a mixture of mono- and divalent bases.

7. The method of claim 6, wherein the monovalent base is selected from sodium hydroxide, potassium hydroxide, and combinations thereof.

8. The method of claim 1, wherein the composition comprising a divalent base is 100% a divalent base.

9. The method of claim 1, wherein the composition comprising a divalent base is present in an amount from about 0.5 to about 1.5 moles per mole of rhamnodilipids.

10. The method of claim 1, wherein the composition comprising a divalent base is present in an amount from about 0.9 to about 1.1 moles per mole of rhamnodilipids.

11. The method of claim 1, wherein the degree of hydrolysis is from about 30% to about 100%.

12. The method of claim 1, wherein the degree of hydrolysis is from about 50% to about 70%.

13. The method of claim 1, wherein the rhamnodilipids are provided in a solvent, and wherein the method further comprises the step of removing the solvent from the rhamnomonolipid hydrolysate.

14. The method of claim 1, further comprising the step of treating the rhamnomonolipid hydrolysate with one or more decolorizing agents.

15. The method of claim 14, wherein the decolorizing agent comprises hydrogen peroxide.

16. The method of claim 1, wherein the separation of the rhamnomonolipid hydrolysate from the by-products is done by a process selected from filtration, precipitation, centrifugation, and combinations thereof.

17. The method of claim 1, wherein the by-products comprise a divalent salt, and the method further comprises the step of separating the rhamnomonolipid hydrolysate from the divalent salt by a process selected from precipitation, filtration, centrifugation, and combinations thereof.

18. The method of claim 1, wherein the by-products comprise b-hydroxy acid and the method further comprises the step of separating the b-hydroxy acid from the rhamnomonolipid hydrolysate via precipitation and filtration.

19. The method of claim 1, wherein the by-products comprise a,b-unsaturated acids and the method further comprises the step of separating the a,b-unsaturated acids from the rhamnomonolipid hydrolysate via precipitation and filtration.

20. The method of claim 1, wherein the by-products comprise b-hydroxy acid and the method further comprises the step of separating the b-hydroxy acid from the rhamnomonolipid hydrolysate via nano-filtration.

21. The method of claim 1, wherein the by-products comprise a,b-unsaturated acids and the method further comprises the step of removing the a,b-unsaturated acids from the rhamnomonolipid hydrolysate via nano-filtration.

22. The method of claim 1, wherein the method further comprises the steps of separating the rhamnomonolipid hydrolysate from the by-products and then adjusting the pH of the rhamnomonolipid hydrolysate to be at least 2.

23. The method of claim 1, wherein the total hydrolysate comprises less than about 10% of by-products.

24. The method of claim 1, wherein the total hydrolysate comprises less than about 5% of by-products.

25. A personal care product comprising the rhamnomonolipid hydrolysate produced in claim 1.

26. A pharmaceutical product comprising the rhamnomonolipid hydrolysate produced in claim 1.

27. The use of the rhamnomonolipid hydrolysate made in claim 1 in a personal care product and/or a pharmaceutical product.
